(19) United States
(12) Patent Application Publication
(10) Pub. No.: US 2025/0228642 A1
(43) Pub. Date: Jul. 17, 2025

(54) Medical lighting device

(71) Applicant: CIVAL MEDICAL GMBH, Tuttlingen (DE)

(72) Inventors: Claudius DIEZ, München (DE); Ingolf DIEZ, Tuttlingen (DE)

(21) Appl. No.: 19/019,948

(22) Filed: Jan. 14, 2025

(30) Foreign Application Priority Data
Jan. 15, 2024 (DE) 10 2024 101 015.0

(51) Int. Cl. A61B 90/35

(52) U.S. Cl. CPC A61B 90/35

(57) ABSTRACT

The present disclosure relates to a medical lighting device. An exemplary embodiment of the medical lighting device has: at least one light source, which is arranged and formed in a such a way to emit light; and at least one lens, which is arranged and formed in such a way to receive the emitted light at least partially and to emit it at least partially in the direction of an operating area to be illuminated. The at least one light source is arranged and formed in such a way to be movable in a direction transversely to a beam path of the light emitted by the at least one light source relative to the at least one lens.

### CROSS-REFERENCE TO RELATED APPLICATIONS

The present application claims the benefit of and priority to German Patent Application No. 10 2024 101 015.0, filed Jan. 15, 2024, the entire contents of which is incorporated herein by reference.

The present invention relates to a medical lighting device.

In medical technology, the illumination of an operating field/operating area for performing an operation is important. To illuminate an operating area during a medical operation, surgical lights are normally used. The operating field in which a surgical procedure takes place must be illuminated in such a way that a surgeon can clearly see the tissue at hand and can move safely in the operating field with the surgical instrument.

In the case of lighting devices known from the state of the art for illuminating an operating field, to illuminate the surface correctly, the pertinent device is held at a position provided for it. The light field for illuminating the operating field is moved along the surface, for example by pivoting the entire lighting device. This is effortful. Moreover, a lot of space is required. This is because a relatively large space must be available to facilitate the pivot movement of the lighting device.

A requirement exists for an improved lighting device. The lighting device should in particular enable illumination of an operating field in a simple manner.

According to a first aspect, a medical lighting device is proposed. The medical lighting device has at least one light source. The at least one light source is arranged and formed in such a way to emit light. The medical lighting device has at least one lens. The at least one lens is arranged and formed in such a way to receive the emitted light at least partially and to emit it at least partially in the direction of an operating area to be illuminated. The at least one light source is arranged and formed in such a way to be movable in a direction transversely to a beam path of the light emitted by the at least one light source relative to the at least one lens.

The medical lighting device can be part of a surgical light or be formed as a surgical light. The medical lighting device can be part of at least one light module of the surgical light. Expressed another way, a surgical light can have one or more medical lighting devices according to the first aspect. For example, a surgical light can have one or more light modules, which has/have in turn one or more medical lighting devices according to the first aspect.

The operating area can generally be any area that is visible to the surgeon. The operating area can be the part, area or section of an operating theatre at which the operation is performed. For example, the operating area can be an operating table or a portion of an operating table or the operating area can comprise the operating table. The operating area can be an area of the operating theatre at which the operation is performed. The medical lighting device or a surgical light having the medical lighting device can illuminate e.g. the entire operating area or can also illuminate just a sub-area of the operating area, e.g. in a focused manner. Let it be stated here purely by way of example that the operating area can be a cover of the patient, e.g. a drape, used in the operation. The operating area can further comprise a point at which the operation is performed.

By means of the medical lighting device, at least one light field can be produced and cast/projected onto at least one area of the operating area/operating field to be lit/illuminated. The at least one light field can be an end of a beam path of the light emitted by the light source. The beam path of the light can have its beginning at the at least one light source.

The light field can be adapted flexibly by a relative movement of the at least one movable/mobile light source relative to the at least one lens. For example, the light field can be changed by the relative movement.

The at least one lens can be arranged along the beam path between the at least one light source and the operating field to be lit/illuminated. By a relative movement of the at least one light source relative to the at least one lens, the at least one light field can be moved along the operating field. In addition or alternatively, the at least one light field can be enlarged or reduced. The at least one lens can be arranged parallel to the at least one light source, in particular a light-emitting surface of the light source. The at least one light source can be arranged and formed in such a way to be displaceable in parallel with respect to the at least one lens. The at least one light source can be arranged and formed in such a way to be movable, in particular displaceable, in particular mechanically or electromechanically, along the beam path and/or transversely to the beam path. The at least one light field can thereby be moved along the operating field to be lit.

The at least one light source can be arranged and formed in such a way to be movable in a direction at least virtually perpendicular to the beam path of the light emitted by the at least one light source relative to the at least one lens. In addition or alternatively, the at least one light source can be arranged and formed in such a way to be movable in a direction at least virtually parallel to an extension direction of the at least one lens. The extension direction can be a direction of a longer or longest side or surface of the lens. The at least one light source can be arranged and formed in such a way to be mechanically or electromechanically movable, in particular displaceable, in the direction transversely to the beam path of the light emitted by the at least one light source relative to the at least one lens.

Due to the movement, in particular the displacement, of the at least one mobile/movable light source relative to the at least one lens, the light field can be moved along the operating field. In addition or alternatively, the light field can be adapted, e.g. enlarged or reduced, by the movement, in particular the displacement, of the at least one mobile/movable light source relative to the at least one lens. The light field can be adapted flexibly thereby. Using the adapted light field, an operating area can simply be illuminated at least partially, in particular in a space-saving manner.

The at least one light source can be arranged or fastened on at least one light source plate. The at least one light source plate can be arranged and formed to be movable in a direction transversely to the beam path of the light emitted by the at least one light source relative to the at least one lens. An extension direction of the at least one light source plate can correspond to a movement direction, for example displacement direction, of the at least one light source. The extension direction of the at least one light source plate can be a direction of a longer or longest side or surface of the light source plate.

A cross section of the medical lighting device or at least of one light module of the medical lighting device can be formed at least virtually round, for example circular or oval. Alternatively, the cross section can be formed polygonal, for example octagonal. The at least one light source can be arranged to be mobile/movable in a radial direction of the at least virtually round cross section.

The at least one lens can be arranged, fastened or formed on at least one lens plate. The at least one lens plate can be arranged and formed stationarily or fixedly relative to the at least one light source. The relative mobility/movability of the at least one light source with regard to the at least one lens can thus be achieved by a mobility/movability of the at least one light source, while the at least one lens can be formed fixedly and thus immobilely/immovably in the medical lighting device. The at least one lens, for example the at least one lens arranged on the at least one lens plate, can be held or fixed by at least one mounting plate. For example, on a first side of the at least one lens plate, a first mounting plate can fix the at least one lens plate and thus the at least one lens, and on a second side of the at least one lens plate, a second mounting plate can fix the at least one lens plate and thus the at least one lens.

The at least one light source can have a plurality of light sources or be formed as a plurality of light sources. The at least one light source can have at least one light diode/light-emitting diode (LED) or be formed as at least one LED. The light source can be formed as a plurality of LEDs or can have a plurality of LEDs.

The at least one lens can have at least one Fresnel lens, in particular a white Fresnel lens, or be formed as at least one Fresnel lens, in particular as at least one white Fresnel lens. The at least one lens can have a plurality of lenses or be formed as a plurality of lenses. Each of the plurality of lenses can be formed as a Fresnel lens or have a Fresnel lens.

The medical lighting device can have at least one screen. The at least one screen can extend in particular at least virtually parallel to the beam path. Expressed another way, the at least one screen can extend at least virtually perpendicular to the extension direction of the at least one lens and/or at least virtually perpendicular to the movement direction of the at least one light source. For example, two screens can be provided per light source. A first screen can extend from a first end of a light source plate at least virtually perpendicular from the light source plate and a second screen can extend from a second end of the light source plate at least virtually perpendicular from the light source plate.

According to a second aspect, a surgical light with a medical lighting device according to the first aspect is proposed. A surgical light according to the second aspect can have one or more medical lighting devices according to the first aspect. For example, a surgical light according to the second aspect can have one or more light modules, which can in turn have one or more medical lighting devices according to the first aspect.

Even if the details described above and below were described with regard to the medical lighting device according to the first aspect, these details can also be realised in a corresponding manner in the surgical light according to the second aspect or vice-versa. Furthermore, all cited elements can respectively be part of the surgical light, i.e. be integrated in the surgical light. For example, the medical lighting device can be a surgical light or part of a surgical light.

Exemplary, specific embodiments are explained below based on the enclosed schematic figures. There are shown:

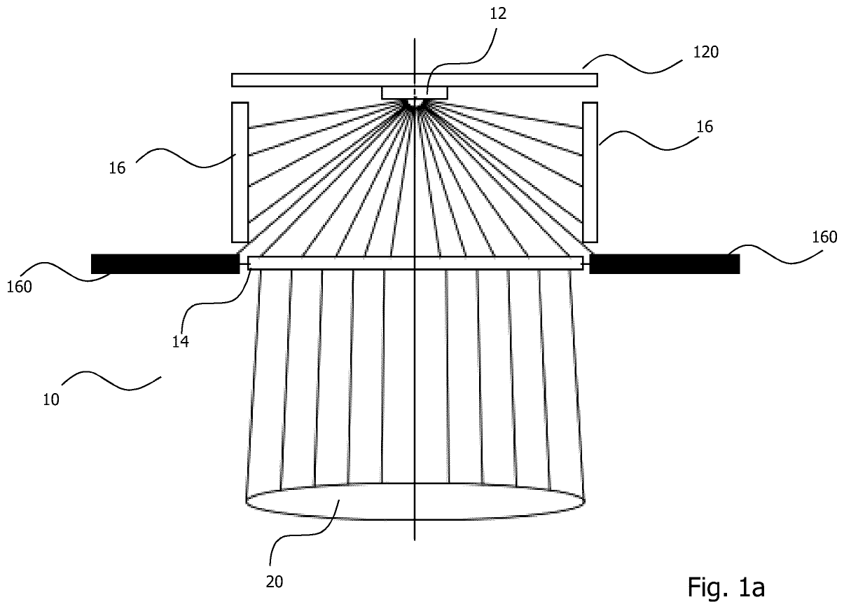

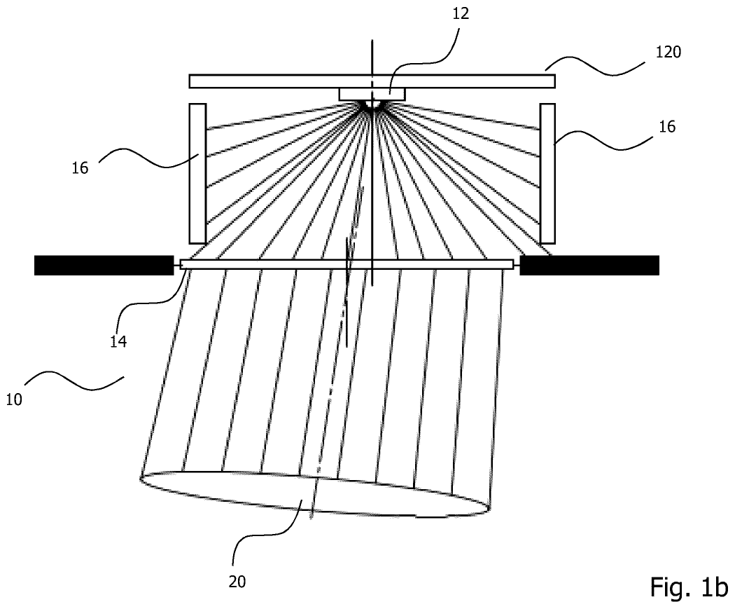

FIG. 1a a schematic representation of an embodiment of a medical lighting device;

FIG. 1b a schematic representation of the embodiment of the medical lighting device from FIG. 1a with a light source moved relative to a depicted lens;

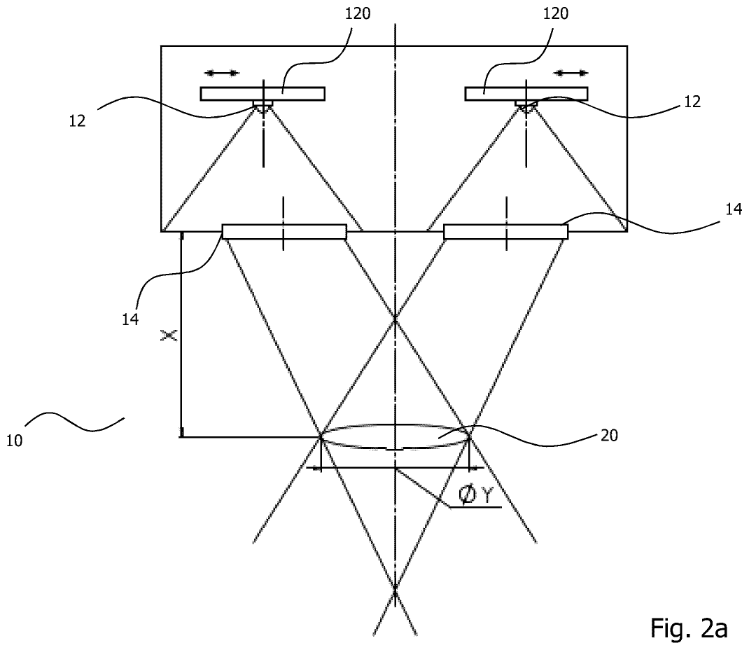

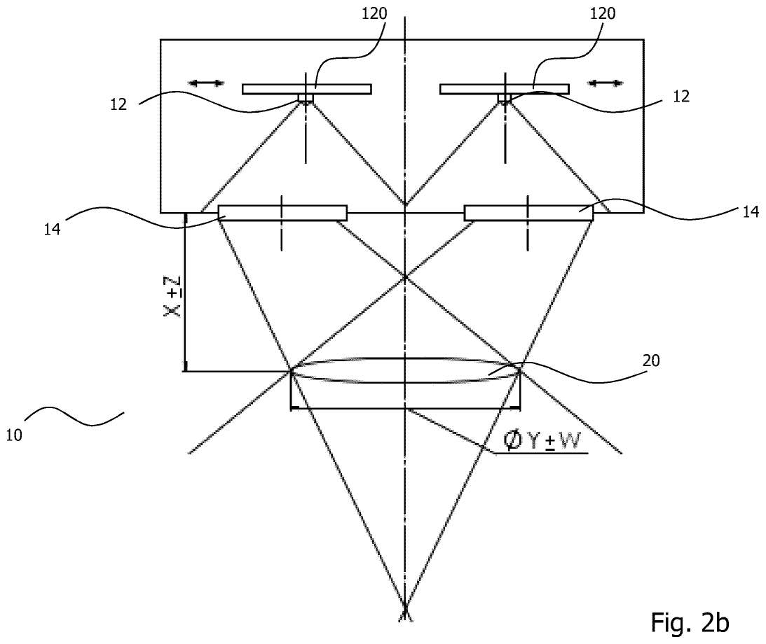

FIG. 2a a schematic representation of an embodiment of a medical lighting device;

FIG. 2b a schematic representation of the embodiment of the medical lighting device from FIG. 2a with light sources moved relative to the depicted lenses;

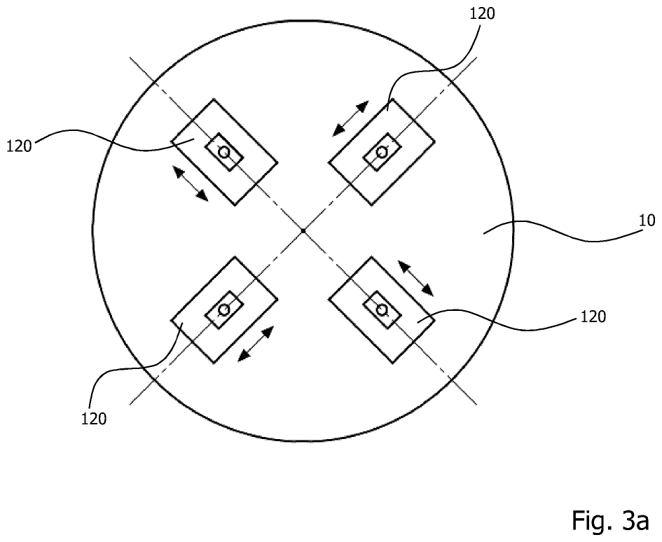

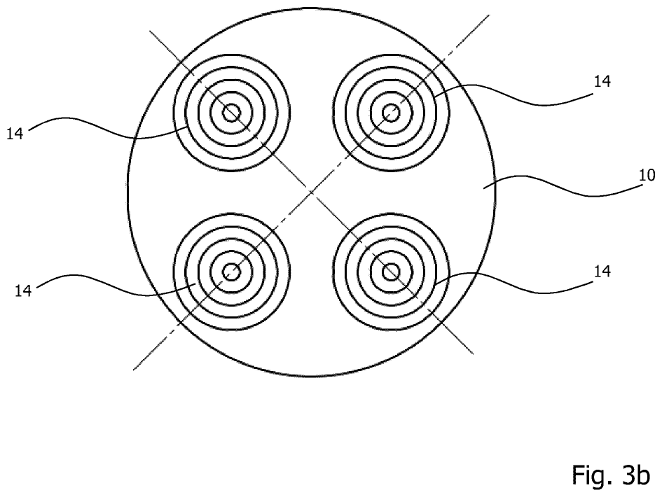

FIG. 3a a schematic representation of an arrangement of several light sources in a medical lighting device; and

FIG. 3b a schematic representation of several lenses in a medical lighting device.

In the following, without being restricted hereto, specific details are described to provide a complete understanding of the present disclosure. It is clear to an expert, however, that the present disclosure can be used in other exemplary embodiments that can differ from the details described below.

FIG. 1a shows, schematically and in a strongly simplified manner, a medical lighting device 10 according to a possible embodiment. The medical lighting device in the example from FIG. 1a is formed as part of a surgical light. In particular, a plurality of medical lighting devices from FIG. 1a can be combined in a suitable manner into a surgical light.

The medical lighting device 10 in the example from FIG. 1a has precisely one light source 12. Even more than one light source 12 can be provided, however. The light source 12 is mounted on a light source plate 120. The light source plate 120 has an extension direction that corresponds in the example from FIG. 1a to a direction running parallel to a horizontal. The medical lighting device 10 in the example from FIG. 1a has precisely one lens 14. More than one lens 14 can also be provided, however. The lens 14 can be arranged or provided in a lens plate. The lens 14 or the lens plate with the lens 14 is held or fixed by way of example at a first end by a mounting plate 160 and at a second end by another mounting plate 160. In the example from FIG. 1a, two mounting plates 160 are provided as an example. In principle, however, any number of mounting plates 160 can be provided to fix the at least one lens 14 or the lens plate with the lens 14.

The medical lighting device 10 further has by way of example two screens 16 as an example of one or more screens. The screens 16 extend as an example respectively from one end of the light source plate 120 perpendicular to (the extension direction of) the light source plate 120.

The light source 12 is arranged and formed in such a way to emit light. The light source 12 forms a beginning of a beam path of the emitted light. In the example from FIG. 1a, the light source 12 emits the light largely downwards, i.e. the beam path runs downwards from the light source 12. The lens 14 is arranged and formed in such a way to receive the emitted light at least partially. In the example from FIG. 1a, a portion of the emitted light falls onto the lens 14. A (smaller) portion of the light falls onto the mounting plates 160. Another portion of the emitted light falls onto the screens 16. The lens 14 emits the received light at least partially in the direction of an operating area to be illuminated. To illuminate the operating area, a light field 20 is produced. The light field 20 can be projected/cast onto an area of an operating field. Thereupon an area of the operating field corresponding to the light field 20 can be illuminated/lit up.

As is to be recognised in FIG. 1b, the light source 12 is arranged movably, for example together with the two screens 16 and the light source plate 120, in a movement direction relative to the lens 14. Stated more precisely, in the example from FIG. 1b, the light source 12 is moved, in particular displaced, by a movement, in particular displacement, of the light source plate 120. The movement direction runs in the example from FIGS. 1a and 1b transversely to the beam path, stated more precisely at least virtually perpendicular to the beam path and/or at least virtually parallel to an extension direction of the at least one lens 14/lens plate and/or at least virtually parallel to an extension direction of the at least one light source plate 120. Due to the movement of the light source 12, in particular due to the displacement of the light source plate 120, the incidence of light onto the lens changes. Moreover, the beam path of the light emitted by the lens 14 changes. The light field 20 at the end of this beam path changes thereby. In the example from FIG. 1b, the light field is pivoted and thereby moved on the operating field. The light field 20 can be moved along the operating field in this way.

FIG. 2a shows schematically and in a strongly simplified manner a medical lighting device 10 according to another possible embodiment.

The medical lighting device 10 in the example from FIG. 2a has two light sources 12 as an example of at least one light source 12. The medical lighting device 10 in the example from FIG. 2a has two lenses 14 as an example of at least one lens 14. In contrast to the example from FIG. 1a, no screens 16 are shown in the example from FIG. 2a, but can also be provided there. Each light source 12 is associated with one of the lenses 14, i.e. the light emitted by the light source 12 is received at least substantially by the associated lens 14.

As in the example from FIG. 1a, the light sources 12 are each arranged on an associated light source plate 120 and are arranged and formed in such a way to each emit light. The light sources 12 form a beginning of a beam path of the emitted light. The lenses 14 are arranged and formed in such a way to receive the emitted light of the associated light source 12 at least partially. The lenses 14 emit the received light at least partially in the direction of an operating area to be illuminated. To illuminate the operating area, a light field 20 arises, as described in relation to FIGS. 1a and 1b. The light field 20 from FIG. 2a results due to superimposition of the respective beam paths of the respective lenses 14.

As is to be recognised in FIG. 2b, both light sources 12 are arranged movably, stated more precisely displaceably, in a movement direction relative to the lenses 14. The movement of the light sources 12, in particular their displacement, takes place in the example from FIG. 2a by way of the movement, in particular displacement, of the light source plates 120. The movement direction in the example from FIGS. 2a and 2b runs transversely to the beam path, stated more precisely at least virtually perpendicular to the beam path and/or at least virtually parallel to an extension direction of the lenses 14. Due to the movement of the light sources 12, stated more precisely due to the displacement of the light source plates 120, the incidence of light onto the lenses changes. Furthermore, the beam path of the light emitted by the lenses 14 changes. The light field 20 changes thereby at the end of this beam path. In the example from FIG. 2b, the light field is enlarged compared with the example from FIG. 2a. For example, a diameter of the light field 20 is enlarged.

The movement of the light source(s) 12 in the examples from FIGS. 1a to 2b can take place in various ways. For example, the light source(s) can be mechanically or electromechanically movable, in particular displaceable, in the movement direction relative to the at least one lens 14.

In FIG. 3a, a possible arrangement is shown schematically of several light sources 12 on a medical lighting device 10, which is formed in the example from FIG. 3a as a surgical light or as a light module of a surgical light. A surgical light can have a plurality of such light modules. In FIG. 3a (likewise in FIG. 3b), the surgical light or a housing of the surgical light 10, stated more precisely a section through the surgical light 10 or a housing of the surgical light 10, is depicted circular as an example, but is not limited to a circular shape. An oval or polygonal shape is also conceivable, for example.

By way of example, four light sources 12 are shown in FIG. 3a as an example of at least one light source 12. The surgical light 10 or the light module of the surgical light 10 in the example from FIG. 3a has a round cross section, stated more precisely a circular cross section as an example. The four light sources 12 are arranged in such a way that they are mobile/movable in a radial direction of the surgical light 10 or of the light module of the surgical light 10. For example, the light sources 12 can each be displaceable or supported slidingly in the radial direction. Expressed another way, the respective movement directions of the light sources 12 run in a radial direction of the surgical light 10 or of the light module of the surgical light 10. Furthermore, the light sources 12 in their starting position have respectively equal spacing to their respectively adjacent light source 12 in a circumferential direction. In the case of the four light sources 12, this means that the light sources 14 are each spaced at an angle of 90 degrees from their respectively adjacent light sources 12.

In FIG. 3b, a possible arrangement of several lenses 14 on a medical lighting device 10 is shown, which is formed in the example from FIG. 3b as a surgical light or as a light module of a surgical light. Four lenses 14 are shown by way of example in FIG. 3b as an example of at least one lens 14.

The four lenses 14 are arranged in such a way that they have respectively equal spacing from their respectively adjacent lens 14 in a circumferential direction. In the case of the four lenses 14, this means that the lenses 14 are each spaced at an angle of 90 degrees from their respectively adjacent lenses 14. Each of the lenses 14 can be formed as a Fresnel lens, in particular a white Fresnel lens. The lenses 14 from FIG. 3b can be arranged in such a way that they can interact with the arrangement of light sources 12 from FIG. 3a, as was described in relation to FIGS. 1a to 2b.

By means of the exemplary embodiments of the medical lighting device shown by way of example, a light field can be adapted in a simple manner.

1. A medical lighting device comprising:
at least one light source, which is arranged and formed in such a way to emit light; and
at least one lens, which is arranged and formed in such a way to receive the emitted light at least partially and to emit it at least partially in the direction of an operating area to be illuminated;
wherein the at least one light source is arranged and formed in such a way to be movable in a direction transversely to a beam path of the light emitted by the at least one light source relative to the at least one lens.

2. The medical lighting device according to claim 1,
wherein the at least one light source is arranged and formed in such a way:
to be movable in a direction at least virtually perpendicular to the beam path of the light emitted by the at least one light source relative to the at least one lens;
and/or to be movable in a direction at least virtually parallel to an extension direction of the at least one lens relative to the at least one lens.

3. The medical lighting device according to claim 1, wherein the at least one light source is arranged and formed in such a way to be mechanically or electromechanically movable, in particular displaceable, in the direction transversely to a beam path of the light emitted by the at least one light source relative to the at least one lens.

4. The medical lighting device according to claim 1, wherein the at least one light source is arranged or fastened on at least one light source plate and the at least one light source plate is arranged and formed to be movable in a direction transversely to the beam path of the light emitted by the at least one light source relative to the at least one lens.

5. The medical lighting device according to claim 1, wherein a cross section of the medical lighting device is formed at least virtually round and the at least one light source is arranged to be mobile/movable in a radial direction of the round cross section.

6. The medical lighting device according to claim 1, wherein the at least one lens is arranged, fastened or formed on at least one lens plate and the at least one lens plate is arranged and formed stationarily or fixedly relative to the at least one light source.

7. The medical lighting device according to claim 1, wherein the at least one light source comprises a plurality of light sources or is formed as a plurality of light sources.

8. The medical lighting device according to claim 1, wherein the at least one light source comprises at least one light diode or is formed as at least one light diode.

9. The medical lighting device according to claim 1, wherein the at least one lens comprises at least one Fresnel lens, in particular a white Fresnel lens, or is formed as at least one Fresnel lens, in particular as at least one white Fresnel lens.

10. The medical lighting device according to claim 1, wherein the medical lighting device comprises at least one screen, wherein the at least one screen extends in particular at least parallel to the beam path.
